(19) United States
(12) Patent Application Publication
(10) Pub. No.: US 2016/0338804 A1
(43) Pub. Date: Nov. 24, 2016

(54) MOUTHPIECE-TYPE INTRAORAL SCANNER

(71) Applicants: VATECH Co., Ltd., Gyeonggi-do (KR); VATECH EWOO Holdings Co., Ltd., Gyeonggi-do (KR)

(72) Inventors: Keun Yeong KIM, Gyeonggi-do (KR); Tae Woo KIM, Gyeonggi-do (KR); Yong Joo YANG, Gyeonggi-do (KR); Sung Il CHOI, Gyeonggi-do (KR)

(73) Assignees: VATECH Co., Ltd., Gyeonggi-do (KR); VATECH EWOO Holdings Co., Ltd., Gyeonggi-do (KR)

(21) Appl. No.: 15/161,019

(22) Filed: May 20, 2016

(30) Foreign Application Priority Data
May 20, 2015 (KR) 10-2015-0070170

(51) Int. Cl. A61C 9/00; A61B 5/00

(52) U.S. Cl. CPC A61C 9/0053; A61B 5/0088; A61B 5/0066; A61C 9/0006; A61B 2562/0233; A61B 2562/04; A61B 2576/02

(57) ABSTRACT

Disclosed is a mouthpiece-type intraoral scanner which is including a mouthpiece-like housing extended along a set of teeth of a patient, a sensor module including a light source built in the housing to light an intraoral structure in a patient's mouth and a sensor installed to detect light reflected from the intraoral structure, and a main processor that is separate from the mouthpiece-like housing, generates three-dimensional information of the intraoral structure based on detection results of the sensor module, and controls the sensor module.

### BACKGROUND OF THE INVENTION

Field of the Invention

The present invention relates to an intraoral scanner and, more particularly, to a mouthpiece-type intraoral scanner structured such that a sensor module including a light source to obtain three-dimensional information of an intraoral structure is built in a mouthpiece-like housing that is arranged to extend along a set of teeth, thereby conveniently obtaining three-dimensional information of an intraoral structure while a patient holds the mouthpiece-shaped housing between his or her teeth.

Description of the Related Art

Generally, an intraoral scanner is an optical device that is put in a patient's mouth to obtain three-dimensional information of an intraoral structure of a patient.

Patent Document 1 discloses an example of an intraoral scanner. The intraoral scanner includes: a casing provided with a window, which is put into a mouth; an image obtaining unit to obtain reference information of an intraoral structure; a three-dimensional information obtaining unit to obtain three-dimensional information of the teeth through the window; and a three-dimensional model generating unit to generate a three-dimensional model of the teeth.

The conventional intraoral scanner causes discomfort or inconvenience to a patient, since angles and positions thereof are adjusted multiple times to obtain three-dimensional information while the intraoral scanner is placed in a patient's mouth.

Furthermore, scanning has to be performed with a patient holding the intraoral scanner, which restricts the patient from performing any other task during scanning. In addition, precision or accuracy of three-dimensional information obtained through the scanning varies in accordance with a patients' ability to steadily hold the scanner.

In addition, the conventional intraoral scanner can scan only a small area corresponding to, for example, only one tooth or two teeth at most at a time. Therefore, it takes a long time to scan the entire area of an intraoral structure with the use of the conventional intraoral scanner.

### DOCUMENTS OF RELATED ART

(Patent Document 1) Korean Patent No. 10-1418403

### SUMMARY OF THE INVENTION

Accordingly, the present invention has been made keeping in mind the above problems occurring in the prior art, and an objective of the present invention is to provide a mouthpiece-type intraoral scanner capable of easily and conveniently obtaining three-dimensional information of an intraoral structure without needing to adjust positions and angles of the scanner that is in a patient's mouth.

Another objective of the present invention is to provide a mouthpiece-type intraoral scanner that can scan a portion or the entire body of an intraoral structure without needing a patient's help or intervention, thus obtaining high-quality three-dimensional information of an intraoral structure without depending on a patient's ability to steadily hold the scanner.

A further objective of the present invention is to provide a mouthpiece-type intraoral scanner that can finish scanning within a shortened period of time.

The above and other objects, features and other advantages of the present invention will be more clearly understood from the following detailed description.

In order to accomplish the objectives of the present invention, there is provided a mouthpiece-type intraoral scanner including: a mouthpiece housing extended along a set of teeth of a patient; and a sensor module including a light source built in the mouth to light an intraoral structure and a sensor to detect light reflected from the intraoral structure, wherein the sensor module scans the intraoral structure while moving through the mouthpiece.

In accordance with at least one embodiment, the sensor may be a plurality of sensors.

In accordance with at least one embodiment, the sensor module may be a plurality of sensor modules by moving in the housing.

A guide rail and a transportation motor for moving the sensor module may be installed in the mouthpiece.

The transportation motor may be a micromotor.

A moving area of the sensor module may vary in accordance with the purpose of use of obtained images.

According to another aspect, there is provided a mouthpiece-type intraoral scanner including: a mouthpiece interposable between a patient's teeth; and a sensor module that is built in the mouthpiece and includes a light source which emits a light beam to an intraoral structure and a plurality of sensors which receives light reflected from the intraoral structure, wherein the sensor module is composed of a plurality of sensor modules that are fixed in different positions in the mouthpiece to scan the intraoral scanner.

The sensor modules may be selectively switched on and off in accordance with the purpose of use of obtained images.

The sensor may be rotated to receive light reflected from teeth.

The mouthpiece may have a shape corresponding to the entire arch, the entire upper jaw, the entire lower jaw, or a portion of the arch.

The sensor module may be equipped with an optical coherence tomography (OCT) module.

The present invention has the following advantages.

First, it is possible to obtain three-dimensional information of a portion or the entire body of an intraoral structure without needing to adjust positions and angles of an intraoral scanner put in a patient's mouth, thus preventing patient discomfort or inconvenience.

Second, it is not necessary for a patient to hold the mouthpiece intraoral scanner in his or her hand during scanning of a portion or the entire body of a patient's intraoral structure. Therefore, it is possible to obtain high-quality three-dimensional information of an intraoral structure without depending on a patient's ability to steadily hold the scanner. Furthermore, a patient and an operator both can freely move during scanning.

Third, since three-dimension information of an intraoral structure is obtained using a mouthpiece equipped with a plurality of sensor modules including a light source, scanning time can be dramatically reduced.

### BRIEF DESCRIPTION OF THE DRAWINGS

FIG. 1 is a diagram illustrating a mouthpiece-type intraoral scanner, put in a patient's mouth, according to a first embodiment of the present invention;

FIG. 2 is a perspective view illustrating the construction of the mouthpiece-type intraoral scanner according to the first embodiment of the present invention;

FIG. 3 is a diagram illustrating an example of a mouthpiece of the mouthpiece-type intraoral scanner according to the first embodiment of the present invention;

FIGS. 4A and 4B are diagrams illustrating movement of the mouthpiece-type intraoral scanner according to the first embodiment of the present invention;

FIG. 5 is a diagram illustrating the internal construction of the mouthpiece-type intraoral scanner according to the first embodiment of the present invention;

FIG. 6 is a diagram illustrating another example of the internal construction of the mouthpiece-type intraoral scanner according to the first embodiment of the present invention;

FIG. 7 is a schematic diagram illustrating a portion of the mouthpiece-type intraoral scanner of FIG. 6;

FIG. 8 is a diagram illustrating a modified example of the internal construction of the mouthpiece-type intraoral scanner according to the first embodiment of the present invention;

FIG. 9 is a schematic diagram illustrating a portion of the mouthpiece-type intraoral scanner of FIG. 8; and

FIG. 10 is a diagram illustrating a mouthpiece-type intraoral scanner according to a second embodiment of the present invention.

### DETAILED DESCRIPTION OF THE INVENTION

Most of the terms used herein have generally known meanings but some terms are defined to have specific meanings by the inventor. In such a case, the terms specifically defined by the inventor should not be understood in accordance with general meanings defined in common dictionaries but be understood in the context of detailed descriptions of embodiments of the present invention.

Hereinafter, technical constructions of the present invention will be described with reference to preferred embodiments and the accompanying drawings.

However, the present invention should not be limited to the embodiments described herein but may be embodied in diverse forms. Throughout the specification, like reference numerals refer to like elements.

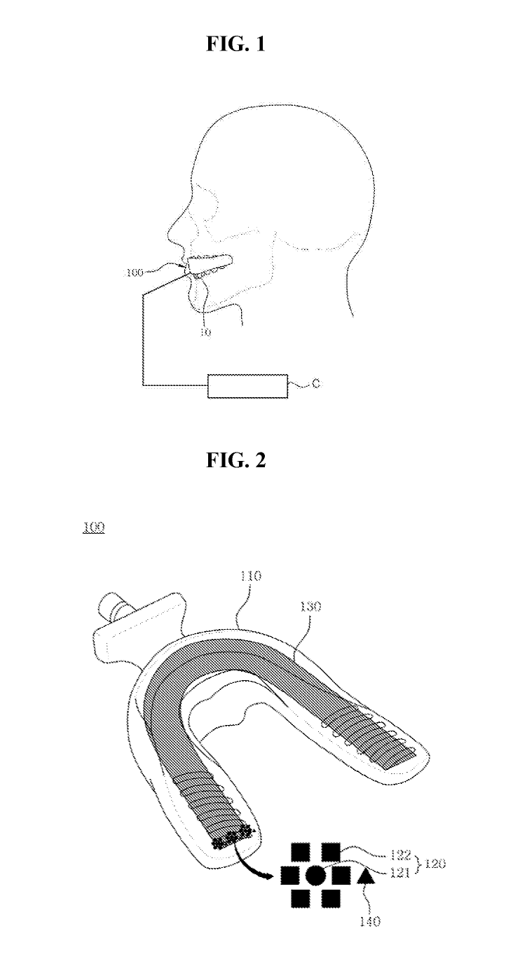

With reference to FIGS. 1 and 2, a mouthpiece-type intraoral scanner 100 according to a first embodiment of the present invention is a device to be arranged along a set of teeth to obtain three-dimensional information of an intraoral structure, namely teeth, of a patient. The mouthpiece-type intraoral scanner 100 includes a mouthpiece-like housing 110, a sensor module 120 installed in the housing 110, and a main processor C separated from the housing 110.

The main processor C includes a signal processing unit that generates three-dimensional information of an intraoral structure based on the detection results of the sensor module 120, and a control unit that controls a moving means described below. The main processor C may have a knob shape and be connected to the housing 110. In this case, when intraoral scanning is performed, a patient may hold the main processor C in his or her hand while the housing 110 is put in his or her mouth.

The housing 110 is put into a patient's mouth to be held between the patient's teeth. At this point, the housing 110 is arranged along the patient's teeth 10.

The housing 110 may be partially or entirely made of a flexible material, but not be limited thereto, thereby reducing patient discomfort attributed to insertion of a foreign body in his or her oral cavity, when the housing 110 is held between a patient's teeth. In addition, the housing 110 may be made of a non-toxic resin that is not harmful to a human being. In addition, at last one surface of the housing 110 facing teeth may be made of a transparent material so that light reflected from an intraoral structure can enter the inside of the housing or light emitted from the inside of the housing can easily travel to an intraoral structure. In addition, the housing 110 of the mouthpiece-type intraoral scanner 100 according to the first embodiment of the present invention may have a shape corresponding to that of the entire arch, the entire upper jaw, the entire lower jaw, or a portion of the arch of a patient.

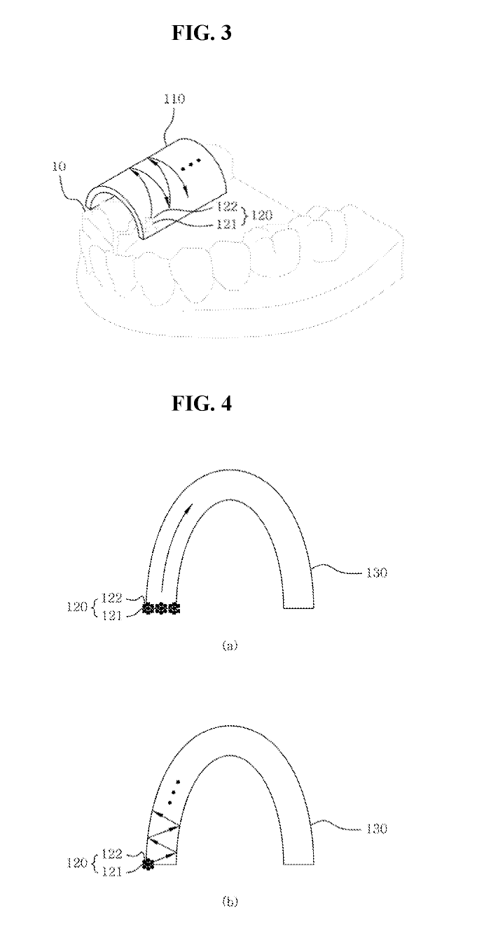

For example, as illustrated in FIG. 3, the housing 110 may have an area as broad as an area of at least two teeth and the housing 110 may take diverse forms.

The sensor module 120 is a device that scans an intraoral structure to obtain three-dimensional information of the intraoral structure, moving through the housing 110. The sensor module 120 includes a light source 121 and a sensor 122.

The light source 121 is a light beam generator that emits a light beam to an intraoral structure.

The sensor 122 is a sensor to detect light reflected from an intraoral structure.

The light source 121 and the sensor 122 may have a size of several micrometers. The specific surface area can be increased by reducing the size of the light source 121 and the sensor 122, which increases a scanning area per scan.

The sensor 122 may be a group of sensors.

In this case, the light source 121 is arranged in the center and the sensors 122 are arranged around the light source 121 in a radial fashion.

The sensors 122 may be rotated to face the light reflected from an intraoral structure. This increases a detection rate of the light reflected from an intraoral structure, resulting in higher-quality three-dimensional information of the intraoral structure.

The intraoral scanner according to the present invention may include a plurality of sensor modules 120 instead of one sensor module. This increases a scanning area per scan, thereby shortening a scanning time it takes to scan the entire area of an intraoral structure.

In the housing 110, a movement path 130 through which at least one sensor module 120 moves is defined. In addition, a moving means for moving at least one sensor module 120 is installed on the movement path 130. The moving means will be described below in detail.

With reference to FIGS. 4A and 4B, the movement path 110 is formed in the housing 110 and serves as a channel through which the sensor module 120 travels. The sensor module 120 can travel along the movement path 110 in the housing 110.

The sensor module 120 may move in an arc-shaped path (see FIG. 4A) or a zigzag-shaped path (see FIG. 4B). However, a direction or pattern of the movement path is not limited thereto.

A moving area of the sensor module 120 varies in accordance with the purpose of use of obtained information.

If the sensor module 120 is controlled to move to obtain an image only within a partial area of the entire arch of a patient, unnecessary use of light and a scanning time can be reduced.

The sensor module 120 may further include an optical coherence tomography (OCT) module (140). In this case, the OCT module 140 moves along the movement path 110 while the sensor module 120 moves.

This construction enables a higher-resolution computed tomographic image with the use of an optical coherence tomography.

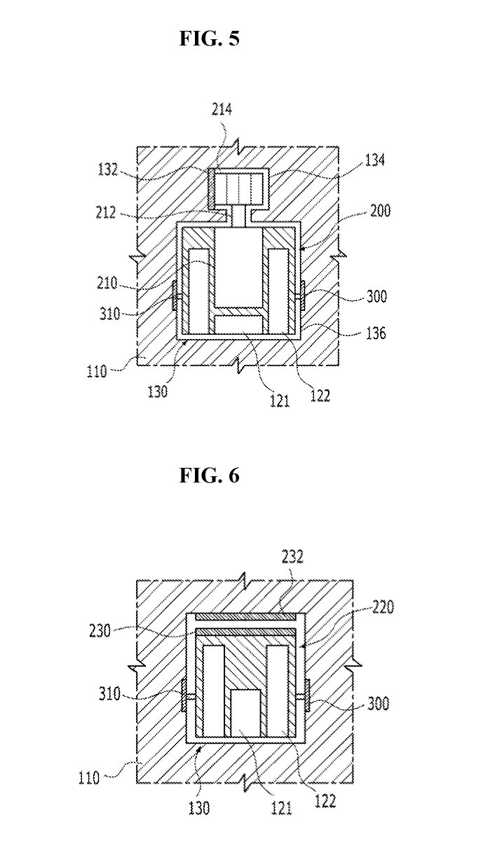

FIG. 5 is a cross-sectional view perpendicular to a longitudinal direction of the housing 110. FIG. 5 illustrates an example of a manner in which the sensor module 120 of the mouthpiece-type intraoral scanner according to the first embodiment moves.

As shown in FIG. 5, the movement path 130 is defined in the housing 110 to extend in a longitudinal direction of the housing 110. A first moving means 200 may be installed in the movement path 130 to move the sensor module 120. The first moving means 200 may include a micromotor 210. The sensor module 120 may be combined with the micromotor 210. A gear 214 is mounted on a motor shaft 212 of the micromotor 210 and a gear train 132 meshed with the gear 214 is installed on a portion of the movement path 130. When the motor shaft 212 is rotated, the micromotor 210 and the sensor module 120 move through the movement path 130.

Preferably, the movement path 130 includes a first movement path 134 for which the gear 214 is provided and a second movement path 136 for which the micromotor 210 and the sensor module are provided. The motor shaft 212 may be installed to extend through the first and second movement paths 134 and 136. The micromotor 210 and the sensor module 120 are connected to a signal line 300 and a brush 310 both of which are arranged to extend along at least part of a surface of the second movement path 136, thereby constantly receiving electric power and communicating data. The gear train 132 is arranged to extend along a portion of the first movement path 134.

Therefore, the sensor module 120 and the micromotor 210 move together to pass through the movement path 130 formed in the housing 110, obtaining three-dimensional information of an intraoral structure.

FIG. 6 is a cross-sectional view perpendicular to the longitudinal direction of the housing 110. FIG. 6 illustrates another example of the manner in which the sensor module 120 of the mouthpiece-type intraoral scanner according to the first embodiment moves.

As shown in FIG. 6, the movement path 130 is defined in the housing 110 to extend along the longitudinal direction of the housing 110. The movement path 130 is provided with a second moving means 220 for moving the sensor module 120. The second moving means 220 may include at least one first electromagnet 230. The first electromagnet 230 may be combined with a portion, preferably an upper end, of the sensor module 120. A second electromagnet 232 is arranged to be spaced from a portion, preferably an upper surface, of the movement path 130, which faces the first electromagnet 230.

Therefore, when voltages and polarities of electric power applied to the first and second electromagnets 230 and 232 are appropriately controlled, the first electromagnet 230 and the sensor module 120 can move, along with the second electromagnet 232, through the movement path 130 in a stepwise manner, thereby obtaining three-dimensional information of an intraoral structure. The first electromagnet 230 and the sensor module 120 are connected to the signal line, brush, etc. arranged along at least part of the movement path 130 to receive electric power and communicate data. The second electromagnet 232 can receive electric power through an additional signal line (not shown), which is arranged to extend along the movement path 130.

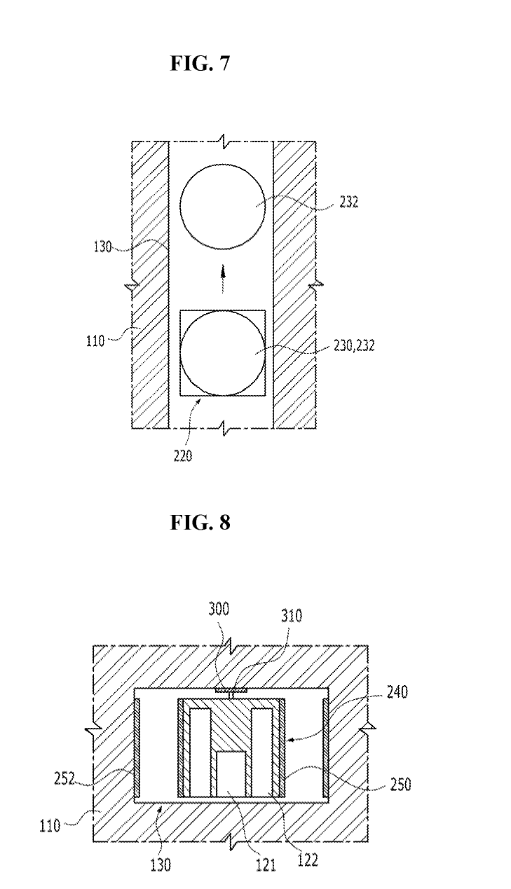

FIG. 7 is a schematic view illustrating a portion of the housing 110 arranged in a longitudinal direction of the housing 110. FIG. 7 illustrates the second moving means 220 that moves through the movement path 130. With reference to FIGS. 6 and 7, voltages and polarities of electric power applied to the first and second electromagnets 230 and 232 are controlled to be switched on and off, so that the second moving means 220 can move in a stepwise manner, along with the second electromagnet 232.

FIG. 8 is a cross-sectional view perpendicular to the longitudinal direction of the housing 110. FIG. 8 illustrates another example of the manner in which the sensor module 120 of the mouthpiece-type intraoral scanner according to the first embodiment moves.

As shown in FIG. 8, the movement path 130 is formed in the housing 110 to extend in the longitudinal direction of the housing 110. A third moving means 240 is installed in the moving path 130 to move the sensor module 120. The third moving means 240 may include at least one third electromagnet 250. The third electromagnet 250 may be attached to at least one surface, preferably to left and right surfaces, of the sensor module 120. Fourth electromagnets 252 are attached to left and right sides of the inside wall surface of the movement path 130 at regular intervals wherein the forth electromagnets 252 on the left and right sides are arranged not to face each other but arranged to alternate with each other.

Therefore, when voltages and polarities of electric power applied to the third and fourth electromagnets 250 and 252 are appropriately controlled, the third electromagnet 250 and the sensor module 120 are moved in a stepwise manner through the movement path 130, while being guided by the fourth electromagnet 252. The sensor module obtains three-dimensional information of an intraoral structure, while moving through the movement path 130. The third electromagnet 250 and the sensor module 120 are connected to the signal line 300 and the brush 310 arranged to extend along at least one surface of the movement path 130, thereby receiving electric power and communicating data. The fourth electromagnet 252 also receives electric power through an additional signal line (not shown) arranged to extend along one surface of the movement path 130.

FIG. 9 is a schematic view illustrating a portion of the housing 110 of FIG. 8, which is viewed in the longitudinal direction of the housing 110. FIG. 9 illustrates the third moving means 240 that moves through the movement path 130. With reference to FIGS. 8 and 9, when voltages and polarities of electric power applied to the third electromagnets 250 and 252 are appropriately controlled, the third electromagnet 250 and the sensor module 120 are moved in a diagonal direction in a stepwise manner. At this point, the fourth electromagnet 252 causes the fourth electromagnet 252 to move in a zigzag fashion.

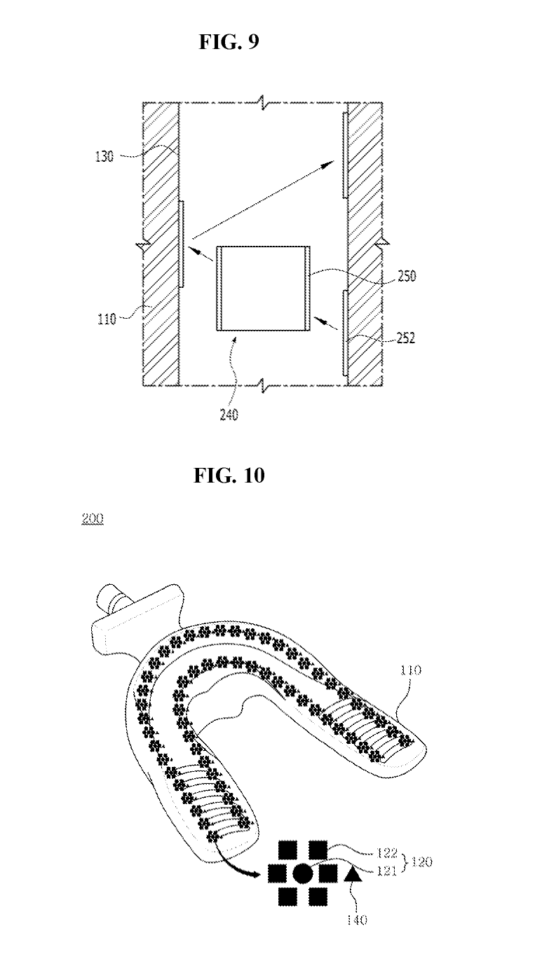

With reference to FIG. 10, a mouthpiece-type intraoral scanner 200 according to a second embodiment of the present invention includes a housing 100 and a sensor module 120. A plurality of sensor modules is fixedly installed in the housing 110.

The housing 110 is a member to be put into a patient's mouth and has a shape conforming to the contour of a patient's teeth. The housing 110 according to the second embodiment is substantially the same as that of the first embodiment.

The sensor module 120 may be a group of sensor modules. Each of the individual sensor modules may be fixed to different positions in the housing 110 and scans a portion of an intraoral structure to produce three-dimensional information of the scanned portion.

Each individual sensor module 120 includes a light source 121 and a sensor 122. This construction is also substantially the same as that of the first embodiment.

Compared to the mouthpiece-type intraoral scanner 100 according to the first embodiment, the scanner 200 is different in that the sensor modules 120 do not move or shift in the housing 110 to scan an intraoral structure, but are fixed in the housing 110 and scan respectively different portions of an intraoral structure at the fixed positions. According to the second embodiment, three-dimensional information of an intraoral structure can be obtained through only one time of scanning.

The mouthpiece-type intraoral scanner 200 according to the second embodiment may further include an OCT module 140. Since this construction is described in the first embodiment, it will not be repeatedly described here.

The sensor modules 120 of the mouthpiece-type intraoral scanner 200 according to the second embodiment of the present invention can be selectively switched on and off, in accordance with the purpose of obtaining three-dimensional information.

In the mouthpiece-type intraoral scanner 200 according to the second embodiment of the present invention, a plurality of sensor modules 120 is fixedly installed over the entire area of the housing 110. Therefore, some of the sensor modules 220 installed at positions supposed to be scanned are switched on and the other sensor modules 220 installed at the other positions are switched off. This control is to avoid unnecessary use of light rays.

As described above, since the mouthpiece-type intraoral scanner according to the present invention scans a patient's intraoral structure using a mouthpiece-like housing equipped with a light source and a sensor, it is possible to minimize movement or adjustment of a scanner in a patient's mouth, thereby reducing patient discomfort or inconvenience and obtaining high-quality three-dimensional information of an intraoral structure, without depending on a patient's ability to steadily hold the scanner.

In addition, since an intraoral structure can be scanned by a plurality of sensor modules at one time, a scanning or radiographing time is dramatically reduced.

Although a preferred embodiment of the present invention has been described for illustrative purposes, those skilled in the art will appreciate that various modifications, additions and substitutions are possible, without departing from the scope and spirit of the invention as disclosed in the accompanying claims.

What is claimed is:

1. A mouthpiece-type intraoral scanner comprising:
a mouthpiece-like housing extended along a set of teeth of a patient;
a sensor module including a light source built in the hosing to light an intraoral structure in a patient's mouth and a sensor to detect a light reflected from the intraoral structure; and
a main processor separated from the mouthpiece-like housing and configured to generate three-dimensional information of the intraoral structure based on detection results of the sensor module and control the sensor module.

2. The mouthpiece-type intraoral scanner of claim 1, wherein the sensor module is a plurality of sensor modules and scans the intraoral structure by moving in the housing.

3. The mouthpiece-type intraoral scanner of claim 1, wherein the sensor is a plurality of sensors.

4. The mouthpiece-type intraoral scanner of claim 1, wherein the sensor module is a plurality of sensor modules fixed at different positions in the mouthpiece-like housing and scanning respectively different portions of the intraoral structure.

5. The mouthpiece-type intraoral scanner of claim 1, wherein the mouthpiece-like housing has a shape corresponding to an entire arch or a part of an arch of the patient, an entire arch or a part of an upper jaw, or an entire or a part of a lower jaw.

6. The mouthpiece-type intraoral scanner of claim 1, wherein the sensor module includes an optical coherence tomography (OCT) module.
